(19) United States
(12) Patent Application Publication
(10) Pub. No.: US 2023/0190481 A1
(43) Pub. Date: Jun. 22, 2023

(54) WHOLE TALUS IMPLANT AND METHOD

(71) Applicant: Paragon Advanced Technologies, Inc., Englewood, CO (US)

(72) Inventors: Gregory J. KOWALCZYK, Little Silver, NJ (US); Luciano BERTOLOTTI, Denver, CO (US); Jesse F. DOTY, Hixson, TN (US); Selene G. PAREKH, Durham, NC (US)

(73) Assignee: Paragon Advanced Technologies, Inc., Englewood, CO (US)

(21) Appl. No.: 18/169,652

(22) Filed: Feb. 15, 2023

Related U.S. Application Data
(63) Continuation of application No. PCT/US2021/046920, filed on Aug. 20, 2021.
(60) Provisional application No. 63/068,182, filed on Aug. 20, 2020.

(51) Int. Cl. A61F 2/42

(52) U.S. Cl. CPC A61F 2/4202; A61F 2002/4207; A61F 2002/4205

(57) ABSTRACT

There is disclosed a talus implant comprising a base having at least one hole and at least one pin. There is also a top comprising at least one hole and at least one pin, wherein the top is configured to be inserted into the base. At least one embodiment comprises a tibial implant comprising at least one post and at least one base coupled to the at least one post. Additionally, there is at least one pad coupled to the at least one base, wherein the at least one pad is selectively insertable into and removable from the at least one base. A method for fabricating a talus and tibial implant is disclosed and also a method for inserting a talus implant and a tibial implant into a patient having a damaged talus joint.

### CROSS REFERENCE TO RELATED APPLICATIONS

This application is a by-pass continuation of PCT International Application No. PCT/US2021/046920, filed Aug. 20, 2021, and entitled Whole Talus Implant and Method, which claims priority benefit under 35 U.S.C. § 119(e) of U.S. Provisional Application No. 63/068,182, filed Aug. 20, 2020, and entitled Whole Talus Implant and Method, which are incorporated herein by reference in their entireties.

### TECHNICAL FIELD

The present invention relates generally to general, podiatric, and orthopaedic surgery related to the replacement of an articulating joint. More specifically, but not exclusively, the present invention relates to the replacement of a total talus in an ankle joint.

### BACKGROUND OF THE INVENTION

At least one embodiment relates to a total talus implant that may be a modular implant. In addition, at least one embodiment comprises a corresponding tibial implant. Furthermore, at least one embodiment involves the process for inserting a talus implant and a tibial implant into a patient.

### SUMMARY OF THE INVENTION

At least one embodiment comprises a talus implant comprising a base having at least one hole and at least one pin. Further, the embodiment may also include a top comprising at least one hole and at least one pin, wherein the top is configured to be inserted into the base. At least one embodiment comprises a tibial implant having at least one post and at least one base coupled to the at least one post. Further, there is at least one pad coupled to the at least one base, wherein the at least one pad is selectively insertable into and removable from the at least one base.

A method for inserting a talus implant and a tibial implant into a patient comprising determining an extent of a damaged talus joint is disclosed. The method may include scanning a contralateral joint, and then creating a mirror image of the contralateral joint. The method may also include printing at least one talus implant comprising at least one base and at least one top as separate parts. The method may also include printing a tibial implant comprising at least one post, and at least one base. Further, the method may include printing at least one pad configured to be coupled to the tibial implant. The method may also include surgically exposing a patient's ankle for removal of a damaged talus and a portion of a damaged tibia. The method may include inserting and securing the tibial implant into a patient's tibia and inserting and securing the base into a patient's ankle, and then surgically closing the wound proximate the patient's ankle.

### BRIEF DESCRIPTION OF THE DRAWINGS

The accompanying drawings, which are incorporated in and constitute a part of the specification, illustrate embodiments of the invention and together with the detailed description herein, serve to explain the principles of the invention. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the invention.

FIG. 1 is an anterior, perspective view of a human foot, in accordance with an aspect of the present invention;

FIG. 2A is a top perspective view of a base of a talus implant in a three dimensional image, in accordance with an aspect of the present invention;

FIG. 2B is a top perspective view of the base of the talus implant of FIG. 2A, in accordance with an aspect of the present invention;

FIG. 3A is a top perspective view of another embodiment of a base of the talus as a three dimensional model, in accordance with an aspect of the present invention;

FIG. 3B is a bottom perspective view of another embodiment of the base of the talus as shown in FIG. 3A, in accordance with an aspect of the present invention;

FIG. 4A is an elevational side view of the base of the talus implant of FIG. 3A, in accordance with an aspect of the present invention;

FIG. 4B is a side, front view of the base of the talus of FIG. 3A, in accordance with an aspect of the present invention;

FIG. 4C is a perspective top view of the base of the talus of FIG. 3A, in accordance with an aspect of the present invention;

FIG. 4D is a top view of the base of the talus of FIG. 3A, in accordance with an aspect of the present invention;

FIG. 5 is a top view of a cross-section of the talus taken along line 1-1 of FIG. 4A, in accordance with an aspect of the present invention;

FIG. 6 is a perspective top view of another embodiment of a base of the talus, in accordance with an aspect of the present invention;

FIG. 7A is a side view of the base of the talus of FIG. 6, in accordance with an aspect of the present invention;

FIG. 7B is a front view of the base of the talus of FIG. 6, in accordance with an aspect of the present invention;

FIG. 7C is a perspective top view of the base of the talus of FIG. 6, in accordance with an aspect of the present invention;

FIG. 7D is a top view of the base of the talus of FIG. 6, in accordance with an aspect of the present invention;

FIG. 8 is a top view of a cross-section of the base taken along line 11-11 of FIG. 7A, in accordance with an aspect of the present invention;

FIG. 9A is a perspective view of a tibia implant and insert, in accordance with an aspect of the present invention;

FIG. 9B is a side view of a base implant for a talus, in accordance with an aspect of the present invention;

FIG. 9C is a top perspective view of a top or crown implant for the talus, in accordance with an aspect of the present invention;

FIG. 9D is a bottom perspective view of the top or crown of the implant for the talus, in accordance with an aspect of the present invention;

FIG. 10 is a bottom view of the crown or top of the talus implant, in accordance with an aspect of the present invention;

FIG. 11 is a top perspective view of the top or crown of the talus implant, in accordance with an aspect of the present invention;

FIG. 12A is a side view of the tibia post implant, in accordance with an aspect of the present invention;

FIG. 12B is a sectional view of the tibia post implant taken along line A-A of FIG. 12A, in accordance with an aspect of the present invention;

FIG. 12C is a bottom view of the tibia post implant, in accordance with an aspect of the present invention;

FIG. 13A is another side view of the tibia post implant, in accordance with an aspect of the present invention;

FIG. 13B is a side view of the tibia post implant, in accordance with an aspect of the present invention;

FIG. 13C is a perspective view of the tibia post implant, in accordance with an aspect of the present invention;

FIG. 13D is a bottom view of the tibia post implant, in accordance with an aspect of the present invention;

FIG. 14 is a perspective view of the tibia post implant, in accordance with an aspect of the present invention;

FIG. 15A is a bottom view of an insert for the tibia post implant, in accordance with an aspect of the present invention;

FIG. 15B is a side view of the insert of FIG. 15A, in accordance with an aspect of the present invention;

FIG. 15C is a cross-sectional view of the insert of FIG. 15A taken along line B-B of FIG. 15A, in accordance with an aspect of the present invention;

FIG. 15D is a side cross-sectional view of the insert of FIG. 15A taken along line A-A of FIG. 15A, in accordance with an aspect of the present invention;

FIG. 15E is an end view of the insert of FIG. 15A, in accordance with an aspect of the present invention;

FIG. 16 is a front view of a foot with the implanted implant including the talus implant and the tibia with the poly insert, in accordance with an aspect of the present invention; and

FIG. 17 is a flow chart for the method of fabricating and implanting a total ankle implant.

### DETAILED DESCRIPTION FOR CARRYING OUT THE INVENTION

Generally stated, disclosed herein are guides, implants, devices, instruments, systems, and assemblies for achieving bone fusion. Further, methods for using the guides, implants, devices, instruments, systems, and assemblies to achieve joint replacement are discussed.

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation and methods, and the aspects, components, features and the like thereof, described herein with respect to the right foot may be mirrored so that they likewise function with the left foot. Further, the implants, devices, instrumentation and methods, and the aspects, components, features and the like thereof, disclosed herein are described with respect to the foot for brevity purposes, but it should be understood that the implants, devices, instrumentation and methods may be used with other bones of the body having similar structures.

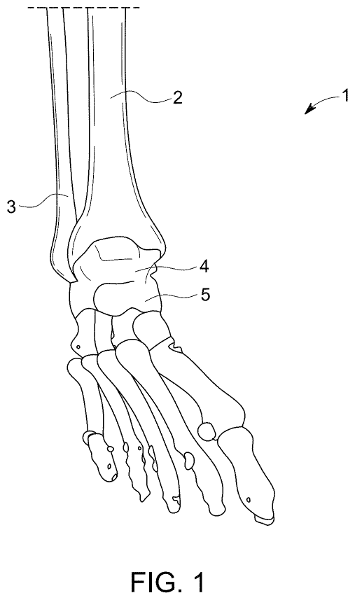

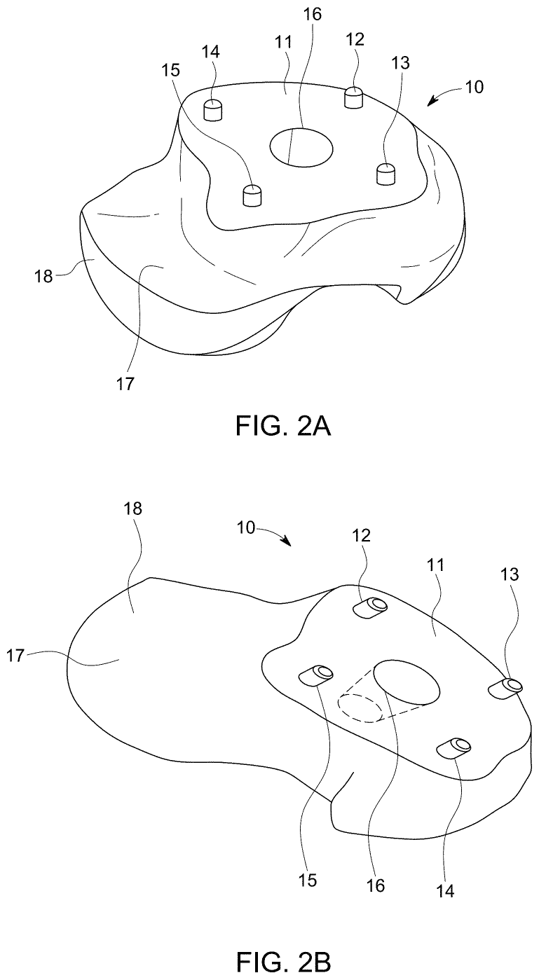

Referring to the drawings, wherein like reference numerals are used to indicate like or analogous components throughout the several views, FIG. 1 is a front view of a foot 1 which includes a tibia 2 a fibula 3, a talus 4, and a navicular 5 among other components. At least one embodiment of the present invention is FIG. 2A is a perspective view of a base of a talus implant in a three dimensional image, the base 10 of the talus implant including a body section 11 as well as pins or posts 12, 13, 14, and 15. There is also a center hole 16 as well as a neck 17 along with a head section 18.

FIG. 2B is a perspective view of the base of the talus implant as shown in FIG. 2A, this view also shows a top surface 11 having pins or posts 12, 13, 14, and 15 along with center hole 16 neck 17 and head 18. This design is configured to receive a head portion or a crown portion to the talus implant.

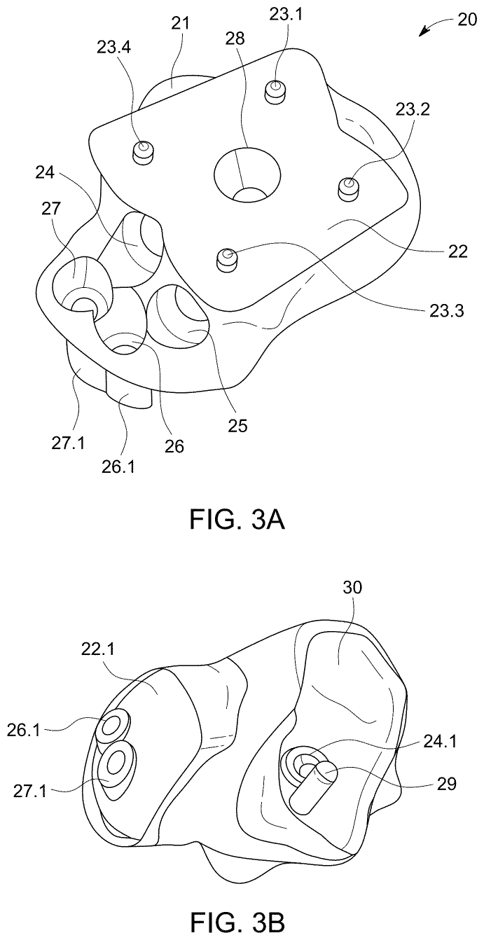

FIG. 3A is a top perspective view of another embodiment of a base of the talus as a three dimensional model. With this design, there is a base which has a body section 21, a top section 22, and four pins or posts 23.1, 23.2, 23.3, and 23.4. There is also a center hole 28 in top section 22. An additional set of holes 24 and 25 are configured to receive pins, while another set of holes 26 and 27 are also configured to receive pins for securing the base within the body and to adjacent bones.

FIG. 3B is a bottom perspective view of another embodiment of the base of the talus as shown in FIG. 3A, in this view there is shown a bottom surface 30, a post 29, an opening 24.1 in bottom section 30, wherein opening 24.1 extends through to hole 24. In addition, there are also additional openings 26.1 and 27.1 in bottom head surface 22.1 as well. These openings allow for the pins to extend therethrough and then secure the base talus to adjacent components.

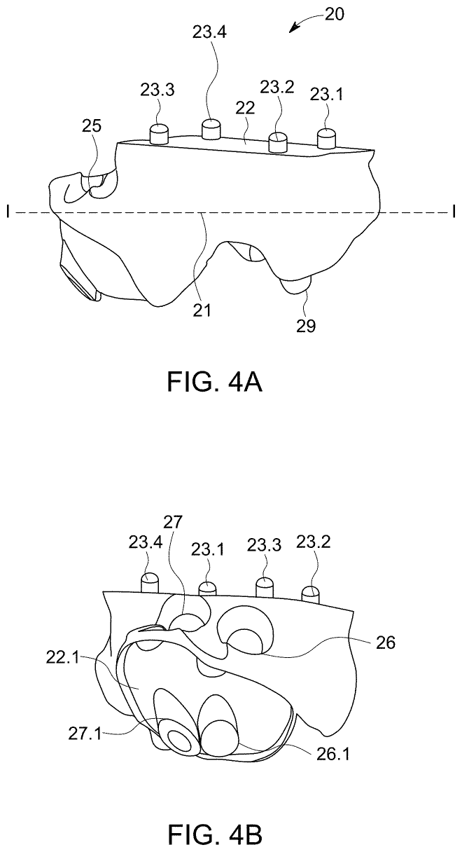

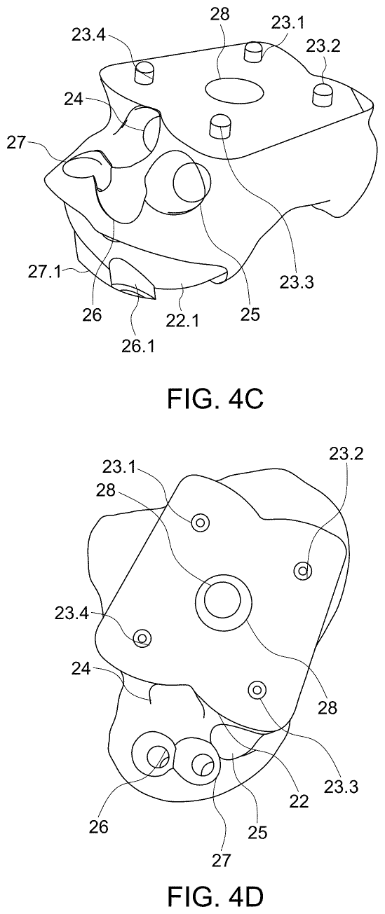

FIGS. 4A, 4B, 4C and 4D are views of the base of the talus implant of FIG. 3A which includes top surface 22, pins 23.1, 23.2, 23.3, and 23.4 as well as a body section 21. There are holes 24, 25, 26, 27 and 28 which are configured to receive pins or inserts, and a bottom head surface 22.1 which include extending cylindrical holes 26.1 and 27.1. In addition, extending out from the bottom surface is a post 29 as well.

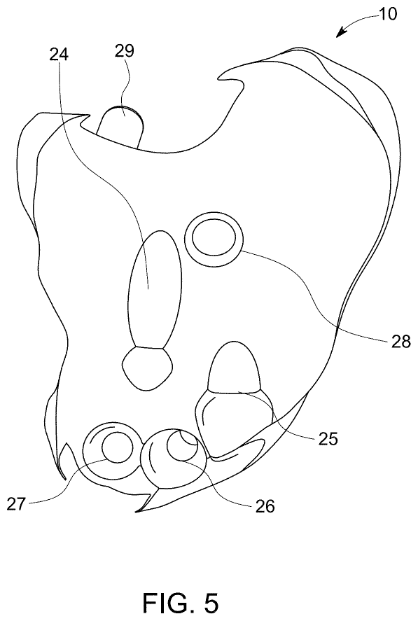

FIG. 5 is a top view of a cross-section of the talus taken along line 1-1 of FIG. 4A. In this view there are shown holes 28, 24, and 25. Hole 28 extends down through a center region of the talus body, while holes 24 and 25 are shown extending transvers to the extension of hole 28.

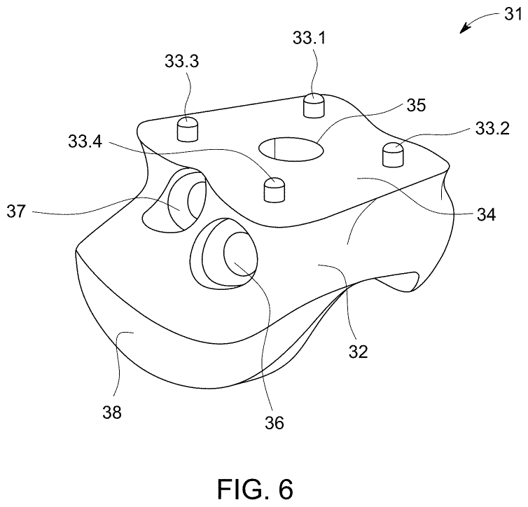

FIG. 6 is a perspective view of another embodiment of a base of the talus 31. This embodiment has a top surface 34, a plurality of pins or protrusions 33.1, 33.2, 33.3, 33.4 and a plurality of holes 35, 36, and 37. Holes 36 and 37 are in a front neck region adjacent to head 38.

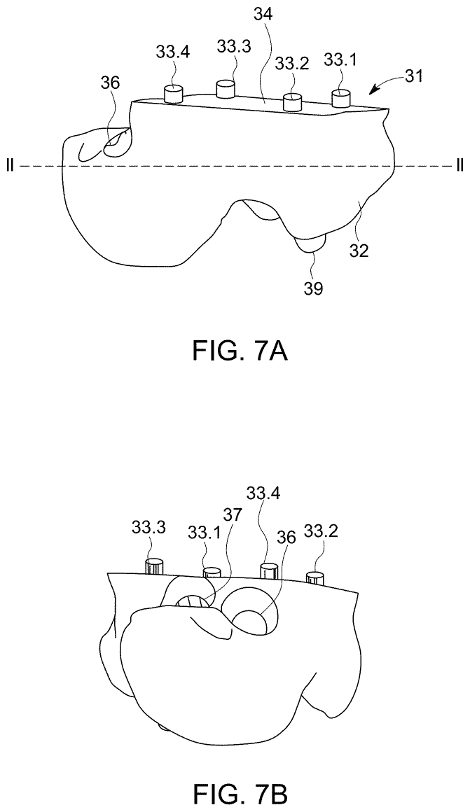

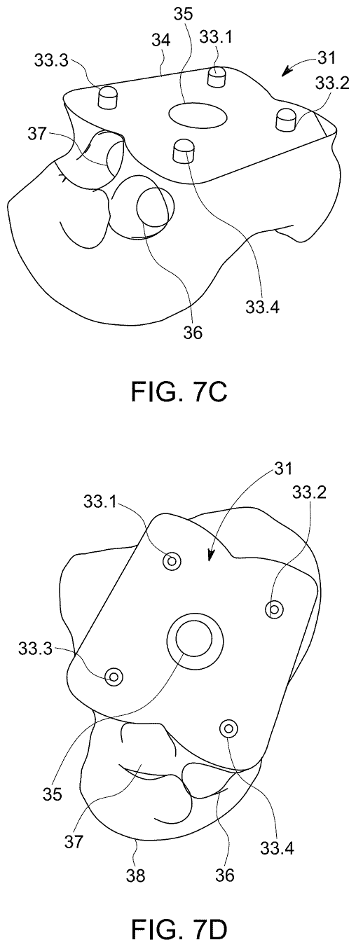

FIGS. 7A, 7B, 7C and 7D also show the plurality of pins/posts 33.1, 33.2, 33.3, and 33.4 which extend up from top surface 34 of body 32. Disposed in top surface 34 of body 32 is hole 35. Further, there are additional holes 36 and 37 extending into body 32 as well.

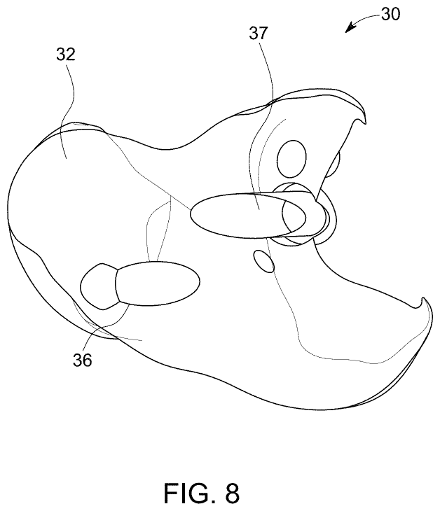

FIG. 8 is a top view of the base taken along line 11-11 shown in FIG. 7A In this view there is shown holes 37 and 36 extending transverse through the body section 32. This extension is transverse to hole 35 as shown in FIG. 7D.

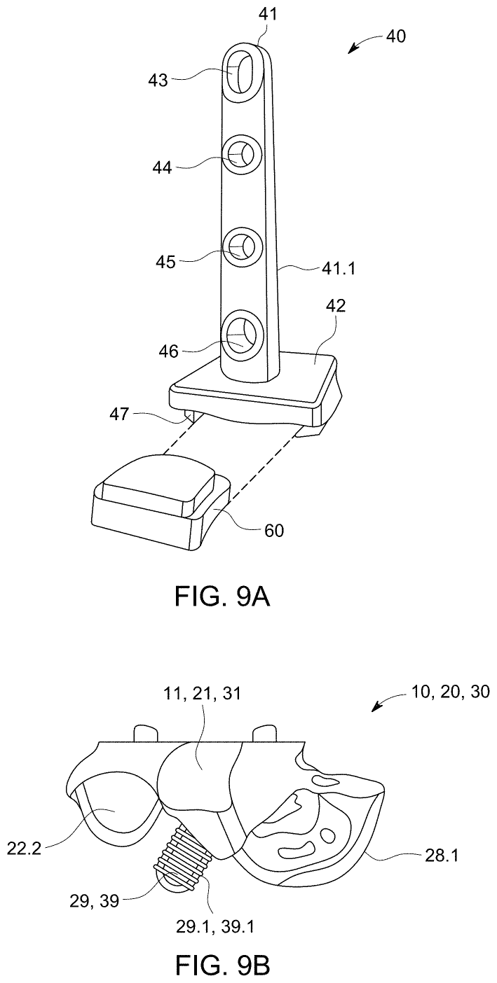

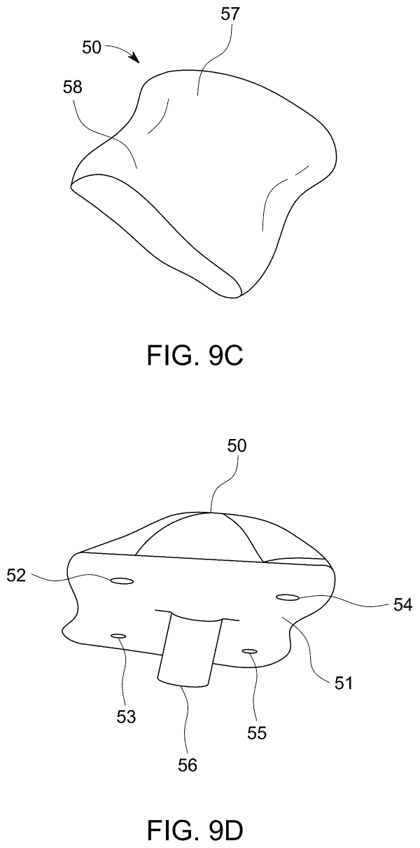

FIG. 9A is a side view of a tibia implant 40 which has a post 41 which is covered in a mesh or screen 41.1. This mesh or screen can be in the form of a honeycomb mesh or screen of varying porosity, wherein the porosity of the screen is greater at the outer regions of the mesh and less as the mesh gets closer to the post section. Holes 43, 44, 45, 46 are positioned in this post 41. Post 41 extends down to base 42. Base 42 has flanges 47 which are configured to receive an insert 60. Insert 60 is configured to insert into post 41 and be fixed to flanges 47 and locked into place with lock mechanism 48.

FIG. 9B is a side view of a base implant for a talus which could be any form of talus implant 10, 20, or 30 having any one of a base 11, 21, or 31. Base 11, 21, or 31 can have any one of a post 29 and or 39 which can be covered in a mesh such as mesh 29.1 or 39.1.

FIG. 9C is a top perspective view of a top or crown implant 50 for the talus which has dome shaped sections 57 and 58. An underside of this crown 50 includes holes 52, 53, 54, and 55 with a post 56 extending down from an underside 51. The post 56 (shown in FIG. 9D) is configured to fit inside of hole such as hole 16, hole 28, or hole 35. Holes 52, 53, 54, and 55 are configured to receive pins or posts 12, 13, 14 and 15 or pins or posts 23.1, 23.2, 23.3 or 23.4 or pins or posts 33.1, 33.2, 33.3, or 33.4.

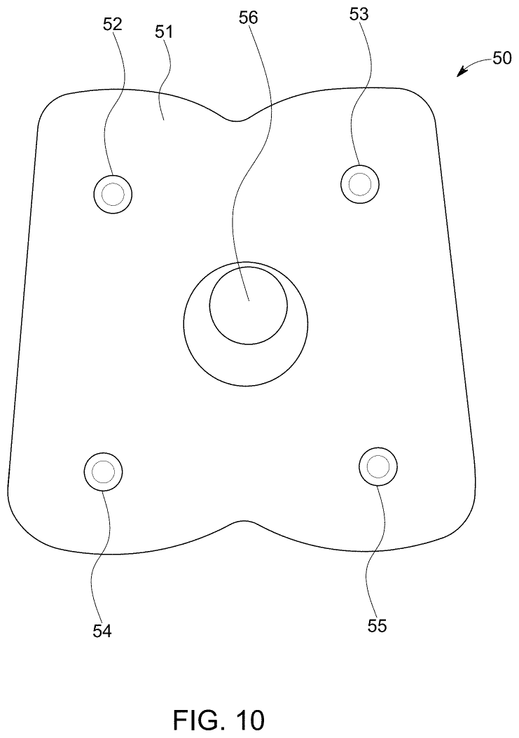

FIG. 10 is a bottom view of the crown or top of the talus implant 50 which shows bottom surface 51, holes 52, 53, 54, and 55 along with post 56. Post 56 is frusto-conical in shape in that it is wider at the base than at its peak and it is formed in a conical or substantially conical shape.

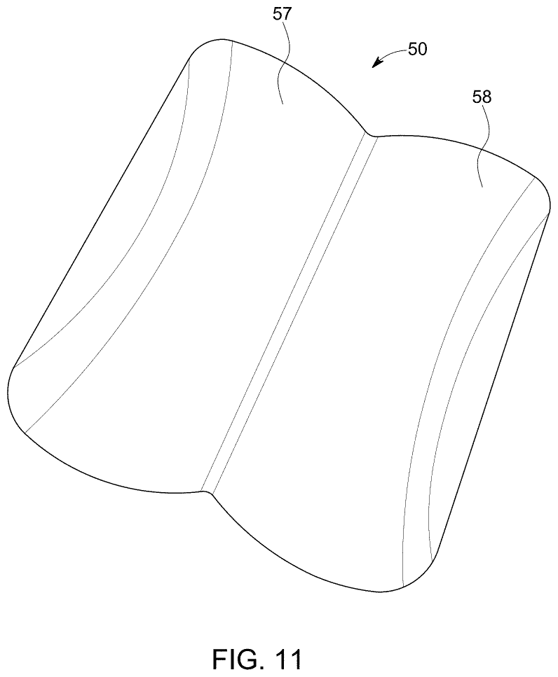

FIG. 11 is a top view of the top of the talus crown implant 50 which has two different crests 57 and 58 positioned on this top surface.

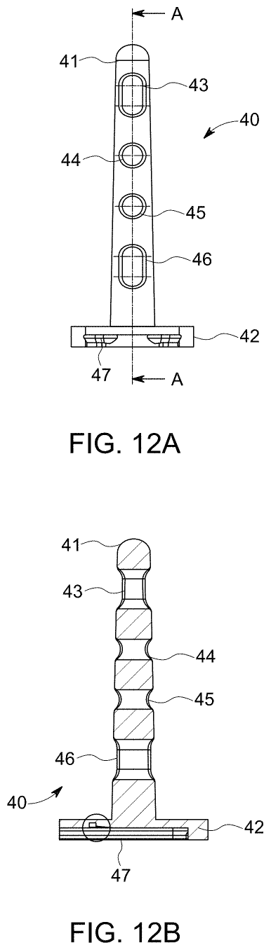

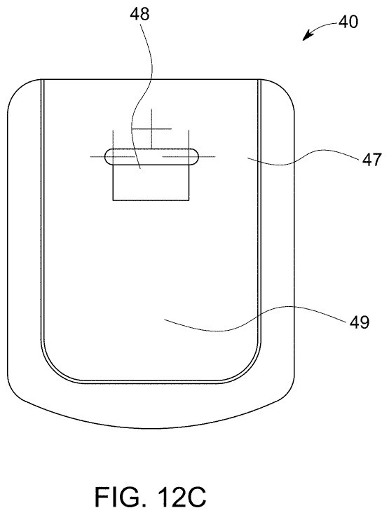

FIG. 12A is a side view of the tibia post implant for a tibia, the post 41 includes holes 43, 44, 45, and 46, along with base 42. Base 42 has flanges 47 which form a slot for the pad.

FIG. 12B is a side sectional view of the tibia post implant for the tibia taken along line A-A of FIG. 12A which shows openings 43, 44, 45, and 46 in post 41. Base 42 includes flanges 47 for accepting the pad. In addition, FIG. 12C is a bottom view of the tibia post implant which shows flanges 47, as well as lock 48 for locking the pad such as pad 60 in place.

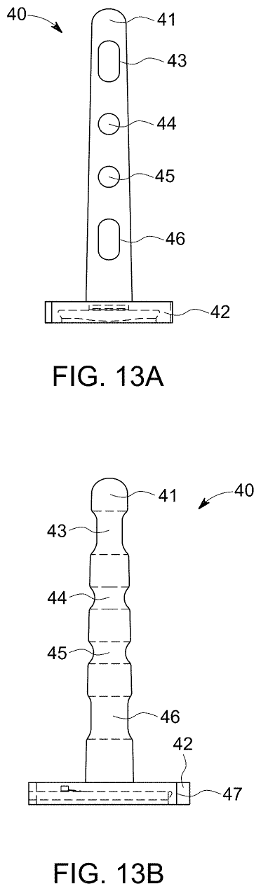

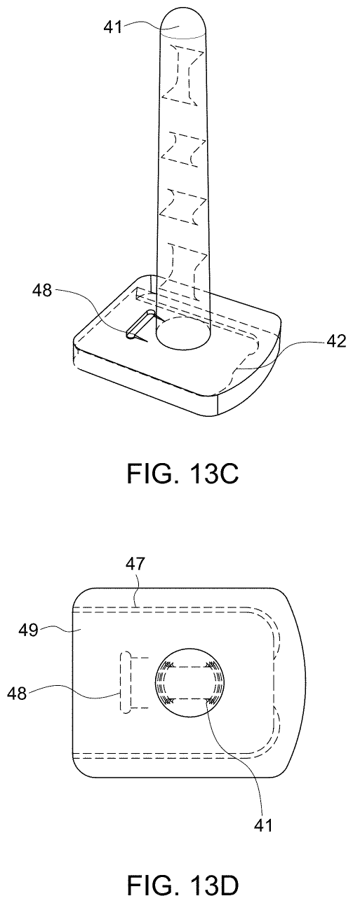

FIGS. 13A, 13B, 13C, and 13D are alternative side views and a bottom view of the tibia post implant 41 which has holes 43, 44, 45, and 46 as well as base 42. Slots 47 are also shown. In particular, FIG. 13D shows lock 48 as well.

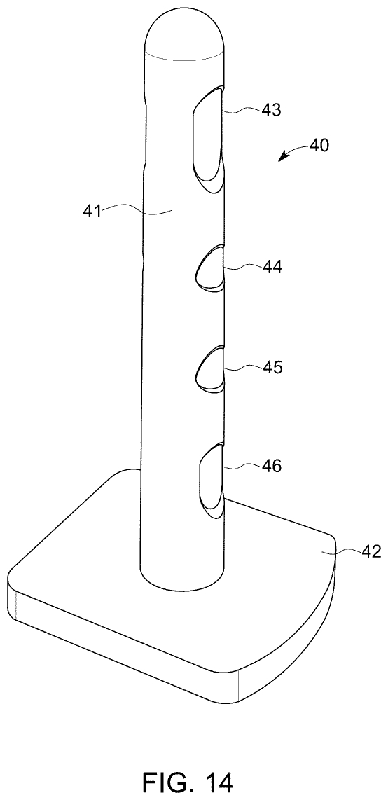

FIG. 14 is a perspective view of the tibia post implant 41 which includes holes 43, 44, 45 and 46, as well as base 42.

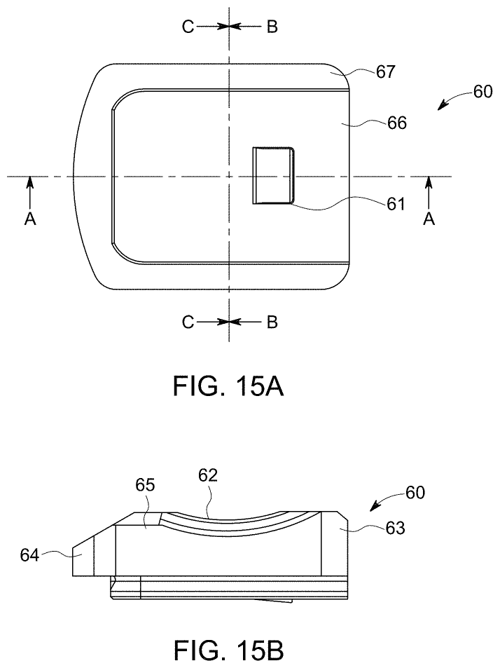

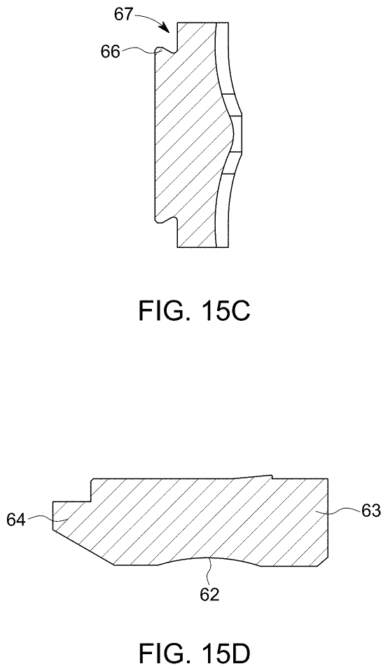

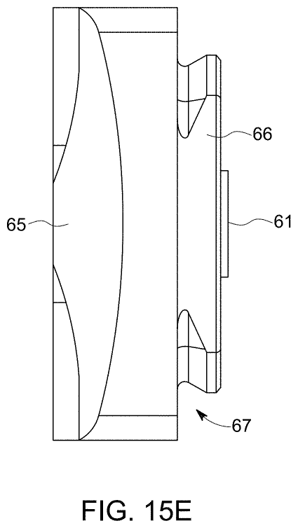

FIG. 15A is a bottom view of an insert or pad 60 for the tibia post implant which may include a slot 67, lock 61 and bottom surface 66. FIG. 15B is a side view of the insert for the tibia post implant 60 which includes a back end 63, a front end 64, and a curved upper surface 62 and a top surface 65 as well. FIG. 15C shows a cross-sectional view which shows flange 66 which extends out from the bottom surface laterally out to form slot 67. FIG. 15D shows a side view taken along line A-A which shows a back end 63, a front end 64, and a top surface 62. FIG. 15E shows an end view which shows bottom 66, along with slot 67, and lock 61 and an end surface 65.

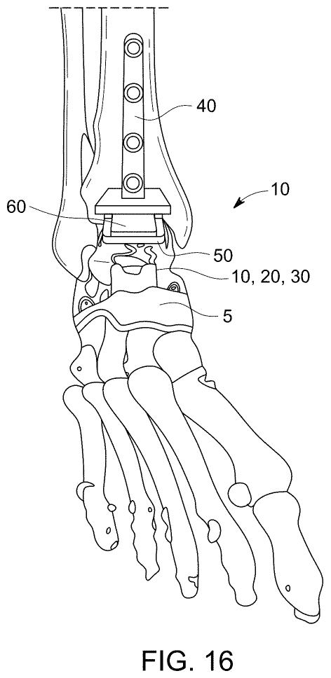

FIG. 16 shows a front view of the tibia implant 40, along with the pad 60. The talus base 10, 20 and/or 30 is shown positioned in the ankle as well as the navicular bone 5 positioned adjacent to the talus base 10, 20 or 30 as well as top 50.

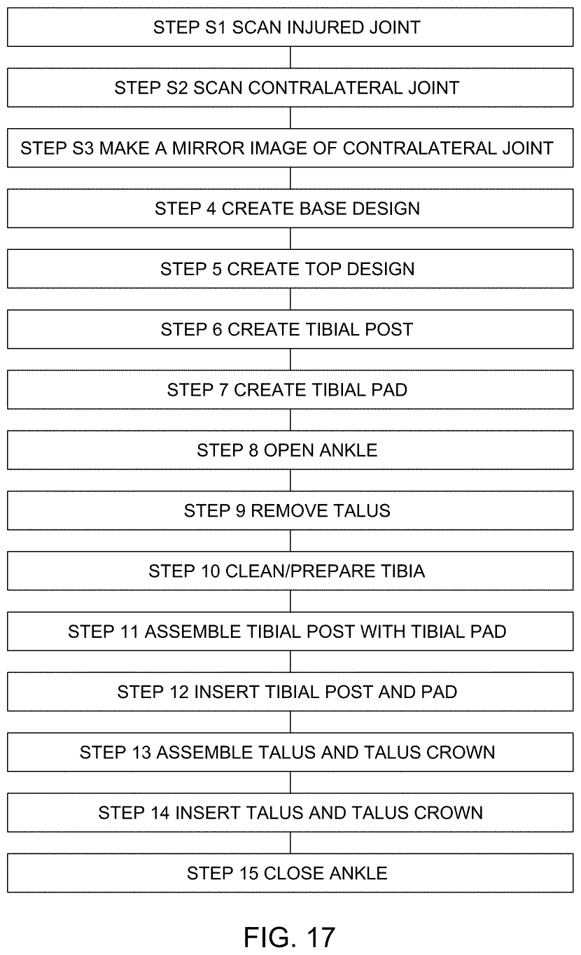

FIG. 17 is a flow chart for the method of fabricating and implanting the tibia implant and the talus implant into a patient. For example, the process starts at S1, wherein the doctor or health care professional scans an injured joint such as a talus. Next, the health care professional may scan a contralateral joint S2. The health care professional may make a mirror image of the contralateral joint S3. This may be done by basically taking a portion of the contralateral joint corresponding to the injured portion of the opposite joint. In this case, when an entire talus is damaged and a tibia is damaged then the entire portion of the talus is scanned and then a mirror image of the contralateral talus is created. A portion of the contralateral tibia may also be scanned and created as well. A base design such as any one of designs 10, 20, or 30 is created S4. A top design such as design 50 is created S5. A tibial post implant is created or printed S6. This type of tibial post implant 40 can then be inserted into a patient's tibia. With the printing of the tibial post, a mesh 41.1 can be printed around the tibial post section 41. A tibial pad may also be printed. S7. The health care professional make an incision over a patient's ankle S8. The damaged talus can be removed from the patient S9. The health care professional may clean and prepare a patient's tibia S10. The health care professional may assemble the tibial post implant with a tibial pad S11. The health care professional may insert the tibial post implant and pad S12. The health care professional may assemble the talus and talus crown S13 and then insert the talus and talus crown into the ankle space S14. The health care professional may then close the incision S15.

Accordingly, the different parts associated with this implant allows for a modular assembly of the tibia post implant 40 as well as the pad 60 along with the top 50 and the talus base 10, 20 and 30 together. Therefore, a health care professional may even during surgery have some flexibility to assembly an appropriate implant for the patient even during surgery.

Accordingly, while at least one embodiment of the present invention has been shown and described, it is to be understood that may changes and modifications may be made thereunto without departing from the spirit and scope of the invention defined in the appended claims.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

Although specific advantages have been enumerated above, various embodiments may include some, none, or all of the enumerated advantages.

What is claimed is:

1. A talus implant comprising:
a base having at least one hole and at least one pin;
a top comprising at least one hole and at least one pin, wherein said top is configured to be inserted into the base.

2. The talus implant of claim 1, wherein said at least one hole is positioned in a central region of the base.

3. The talus implant of claim 1, further comprising at least one additional hole configured to receive at least one pin or post for securing the base in a bone in a patient's leg.

4. The talus implant of claim 1, wherein the base comprises a top surface, and wherein said at least one pin comprises a plurality of pins.

5. The talus implant of claim 4, wherein the top comprises a plurality of holes, wherein the plurality of holes are configured to receive the plurality of pins.

6. The talus implant of claim 1, wherein the top comprises a plurality of holes, and at least one pin.

7. The talus implant of claim 1, wherein the top comprises at least one dome shaped segment.

8. The talus implant of claim 7, wherein the top comprises at least two dome shaped segments.

9. The talus implant of claim 8, wherein the top has a pin, wherein the pin is frusto conically shaped.

10. The talus implant of claim 1, wherein the base comprises a plurality of holes with at least one hole extending through a central region of the base, and at least one additional hole extending substantially transverse to the at least one hole.

11. The talus implant of claim 1, wherein the base further comprises a mesh region, wherein the mesh region comprises varying porosity, and wherein the mesh region extends across a portion of an outer surface of the base.

12. The talus implant of claim 11, wherein the mesh extends across said at least one pin on the base.

13. A tibial implant, comprising:
at least one post;
at least one base coupled to said at least one post; and
at least one pad coupled to the at least one base, wherein the at least one pad is selectively insertable into and removable from the at least one base.

14. The tibial implant of claim 13, wherein the at least one post further comprises at least one mesh region comprising at least one mesh.

15. The tibial implant of claim 14, wherein the at least one mesh region comprises a mesh having variable porosity.

16. The tibial implant of claim 15, wherein the mesh comprises a plurality of cells configured as a honeycomb pattern.

17. The tibial implant of claim 13, wherein the at least one base has at least one flange and at least one lock.

18. The tibial implant of claim 17, wherein the at least one pad includes at least one slot and at least one lock, wherein the at least one slot on the pad is configured to fit into the at least one flange on the base, and wherein the lock on the base is configured to lock with the lock on the pad.

19. A method for inserting a talus implant and a tibial implant into a patient, comprising:
determining a damaged talus joint;
scanning a contralateral joint;
creating a mirror image of the contralateral joint;
printing at least one talus implant comprising at least one base and at least one top as separate parts;
printing a tibial implant comprising at least one post, and at least one base;
printing at least one pad configured to be coupled to the tibial implant;
making an incision over a patient's ankle for removal of a damaged talus and a portion of a damaged tibia;
inserting and securing the tibial implant into a patient's tibia;
inserting and securing the base into a patient's ankle; and
closing the incision over the patient's ankle.

20. The method of claim 19, wherein the inserting and securing of the tibial implant further comprises inserting both a tibial post and a pad which is coupled to the tibial post into the patient.
